(19) United States
(12) Patent Application Publication
(10) Pub. No.: US 2008/0065113 A1
(43) Pub. Date: Mar. 13, 2008

(54) Suture cutter and remover

(76) Inventor: Dean W. Smith, Thousand Oaks, CA (US)

Correspondence Address: STETINA BRUNDA GARRED & BRUCKER, 75 ENTERPRISE, SUITE 250, ALISO VIEJO, CA, 92656, US

(21) Appl. No.: 11/519,692

(22) Filed: Sep. 11, 2006

(51) Int. Cl. A61B 17/10

(52) U.S. Cl. 606/138

(57) ABSTRACT

A suture cutter comprises a tip portion integrally formed with or detachably removable from a handle portion. The handle portion is sized and configured for manipulation by a human hand. The tip portion includes at least one cutting blade disposed within an interior thereof and which faces an opening formed in a tip perimeter. A probe portion is disposed on at least one side of the opening to engage a suture to be severed and removed. The cutting blade preferably has a slightly arcuate shape. The tip portion may further include a remover portion formed along the tip perimeter adjacent the opening and which is preferably sized and configured to grasp a suture for removal thereof following severing of the suture.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present invention relates generally to surgical instruments and, more particularly, to a uniquely configured suture cutting tool that may include a suture removal mechanism and which may be economically mass-produced as a unitary structure to justify disposal of the tool after a single use.

The use of specialized tools for removing sutures or stitches is well known in the art. For example, U.S. Pat. No. 234,981 issued to Harris et al. (the Harris reference) discloses a combined reed hook and picking comb for use by weavers. The Harris device includes a handle having a picking comb that is extendable out of an end of the handle and which may be retracted into the handle when unused. The device includes a blade which is pivotably encased within the handle and which may be drawn outward similar to an ordinary pocket knife for use as a knife blade.

Although the device of the Harris reference is indicated as being suitable for carrying in a vest pocket due to the pivoting blade, the Harris device possesses several drawbacks associated with overall safety and ease of use as a tool for cutting and removing stitches or sutures. More specifically, because of the width of the blade, the Harris device as understood is undesirable for use as a suture cutting tool due to difficulties in inserting the tool within a suture that is sewn into a patient's skin. As is known in the art, it is necessary to insert the tool beneath the suture so that the suture loop may then be severed and removed. Furthermore, the exposed blade of the Harris device presents a risk of injury to the user should the user's hand slip and contact the sharpened steel blade. The patient is also at risk of injury due to general risks posed by the use of the sharpened blade in close proximity to delicate tissue.

U.S. Pat. No. 3,212,187 issued to Benedict (the Benedict reference) discloses a disposable suture cutting and removing blade which includes a blade formed of one piece metal and which is curved or bent transversely at a point intermediate its ends so as to create a handle portion and a cutting edge extending from the bend. The Benedict device may be utilized by slipping the cutting edge underneath the suture in order to sever the suture. A notch formed in a side of the blade opposite from the cutting edge is configured to engage a knot of a suture after severing to facilitate removal of the suture. Although the Benedict device provides a simplified and, therefore, more economically-produceable suture removing tool as compared to the Harris device, the exposed cutting blade of the Benedict device presents the same risks of injury to patient and/or user.

U.S. Pat. No. 3,600,806 issued to Naccash (the Naccash reference) discloses a suture removing instrument having a curved needle that includes a sharp inner edge or blade and a blunted point. An opposite end of the needle includes a flat area to act as a handle for manipulation of the needle by a user. Use of the instrument to remove sutures includes grasping the flat area and inserting the blunted point between the suture and the skin surface. The instrument is then moved beneath the suture such that the inner edge contacts and severs the suture. Although the Naccash device possesses an advantage due to its small size which facilitates insertion of the needle between the suture and the skin surface with little patient discomfort, the relatively small flat area provide an inadequate mechanism by which a user may comfortably grasp the needle which, in turn, limits the ease with which the needle may be manipulated.

U.S. Pat. No. 4,384,406 issued to Tischlinger (the Tischlinger reference) discloses a combination suture cutting and removing instrument which includes a handle having a head portion extending longitudinally outwardly therefrom. The head portion is curved in a crescent shape and has a flat end. The head portion includes a suture cutter blade positioned across the concave portion of the head. The head portion further includes a V-shaped notch whereby the flat end may be used to bring the cutter into engagement with the V-shaped notch to enable the suture knot to be urged upwardly for removal. Although the Tischlinger reference possesses certain improvements due to the inclusion of an elongate handle to facilitate manipulation of the tool as compared to the above-mentioned devices, the generally exposed nature of the cutter blade presents the same risks of injury to patient and user as was earlier mentioned.

U.S. Pat. No. 3,879,846 issued to Allen (the Allen reference) discloses a surgical suture cutter and remover formed of spring-like material into a forceps-like shape. The Allen reference also includes a longitudinal suture cutting element. The cutting element extends between a pair of arms of the forceps and is anchored to one of the arms as well as to a U-shaped portion of the forceps body. In order to remove sutures, a user such as a surgeon forces one of the arms underneath a suture loop and works the cutting edge forward in the loop in order to sever the loop. After severing the suture loop, the Allen device may then be utilized as a tweezers to pull the severed loop from the patient tissue for removal thereof. Although the Allen device is understood to provide a safer mechanism for suture removal due to the positioning of the cutting element between the U-shaped arms of the forceps, the relatively large cross-sectional area of the arm to be inserted under the suture loop may cause undue patient discomfort as the loop is stretched prior to severing by the cutting element.

U.S. Pat. No. 4,098,157 issued to Doyle (the Doyle reference) discloses a suture removal instrument including a forceps that is integrally molded at one end of the handle. A curved blade is rigidly secured to an opposing end of the handle. The cutting blade is understood to be substantially thin and flat and having a sharp concave cutting edge which widens to a non-cutting convex bearing edge. The cutting and bearing edges converge to a sharp point. Due to the generally elongate handle which may be provided with gripping features, manipulation of the Doyle device is improved as compared to the above-referenced devices. However, because of the direct exposure of the curved blade and the sharp point provided on an end thereof, the Doyle device as a suture removal tool is understood to present a high risk of danger of injury to both patient and user.

U.S. Pat. No. 4,246,698 issued to Lasner (the Lasner reference) discloses a suture remover comprising a shearing portion having a cutting edge and a blade having a cutting section. Configured similar to conventional scissors, the cutting edge and cutting section are pivotally connected to one another. The shearing portion further includes a contact area which overlies the cutting edge which is adapted to a portion of the cutting edge. The cutting edge is adapted for insertion between tissue and a suture for shearing contact thereof followed by gripping of the suture with the contact area for removal of the suture. Although the suture remover of the Lasner reference provides a reduced risk of injury due to the scissors-like configuration of the cutting section, the bulkiness of the cutting section may present difficulties in insertion thereof between the tissue and the suture which may cause undue patient discomfort.

U.S. Pat. No. 4,494,542 issued to Lee (the Lee reference) discloses a suture cutting instrument that is adapted to lift individual sutures from the tissue or skin of a patient. The Lee instrument comprises an elongate body having a generally straight handle at one end and a pair of tines formed on an opposite end. The tines are upturned and are of unequal length and having blunt edges defining a narrow V-shaped slot therebetween. The longer one of the tines is insertable between the suture and the patient's skin and is adapted to engage a knot of the suture for subsequent removal by lifting the knotted end held between the two tines. Although the Lee device appears to provide a relatively safe tool for removing sutures, there is no provision for cutting of sutures which necessitates the use of a separate suture-cutting tool.

U.S. Pat. No. 4,848,341 issued to Ahmad (the Ahmad reference) discloses a suture cutting instrument comprising a blade provided in a housing having a generally C-shaped configuration. The blade is integrated into the non-operative end of a suture-tying forceps or at one of the ends of a handle of a needle holder. The blade has a cutting edge which faces an open side of the C-shaped housing. The blades are V-shaped in order to engage and sever a suture. Although the device disclosed in the Ahmad reference appears to provide a generally safe device for cutting sutures due to the non-exposure of the cutting edge, the device is not understood to provide any means for removal of the severed suture.

As can be seen from the above described devices, there exists a need in the art for a suture cutting and removal instrument wherein the cutting element is not exposed in order to reduce the risk of injury to a user (i.e., a doctor and/or surgeon) as well as reduce the risk of injury to a patient. Furthermore, there exists a need in the art for a suture instrument that conveniently integrates both cutting and removal functions into a single unitary structure. In addition, there exists a need in the art for a combination suture cutting and removal instrument that may be economically mass-produced such that the instrument may be disposed of after a single use in order to reduce the risk of infection.

### BRIEF SUMMARY

The above-mentioned deficiencies associated with suture cutting and removal tools of the prior art are specifically addressed and alleviated by the combination suture cutter and removal tool of the present invention. More particularly, the suture cutter is specifically adapted to combine cutting and removal of stitches and/or sutures in a disposable unitary combination medical instrument.

In its broadest sense, the suture cutter includes a non-exposed cutting blade which is formed within an interior of a tip portion of the suture cutter in order to reduce the risk of injury to a patient or user. The suture cutter comprises an elongate, generally straight handle portion attached to or integrally formed with the tip portion. The handle portion is preferably sized and configured for gripping and manipulation by a human hand. In this regard, the handle portion has a generally rounded cross-sectional shape and is of a suitable length to allow a user to conveniently grasp and axially rotate the suture cutter to any desired operative orientation.

The tip portion may have a generally flattened, planar cross-sectional shape and may have a generally rounded profile. More particularly, the tip portion has a relatively thin cross-sectional area relative to the cross-sectional area of the handle portion in order to facilitate insertion thereof between a suture and a patient's skin. The thin profile is therefore configured to minimize patient discomfort which is otherwise caused by the forcing of relatively large instruments under the suture which requires depressing the patient's skin in an area adjacent to a sutured wound.

The tip portion preferably has a rounded tip perimeter with an opening formed therein on a side opposite the handle portion. An exteriorly accessible cutting blade is disposed within the tip portion adjacent the opening and is generally accessible via the opening. Advantageously, the unique arrangement of the cutting blade within the tip portion eliminates or at least reduces the risk of injury to a patient or user due to the non-exposed nature of the cutting blade. The interior location of the cutting blade thereby reduces the risk of accidental contact between the cutting blade and the user's hand or fingers as may otherwise occur if the hands slip off the handle portion during suture cutting. In addition, contact between the cutting blade and the patient skin is also minimized due to the interior placement of the cutting blade.

The cutting blade preferably has an annular or arcuate shape although other shapes are contemplated. In this regard, the cutting blade may be configured in a general horseshoe-like or U-shape in order to facilitate placing the cutting blade into direct contact with the suture after the suture passes through the opening in the tip perimeter. The cutting blade may be integrally formed with the tip portion or the cutting blade may be separately formed as a metallic insert that is retained by a receiving slot formed within the tip portion.

The tip portion may further include a pair of probe portions on opposite sides of the opening. Such probe portions may extend from each side of the tip perimeter and are preferably configured to extend slightly past opposing ends of the cutting blade by a pre-determined amount. In this regard, each of the probe portions preferably extends past a respective one of the cutting blade ends by an amount no greater than about 1000 microns and preferably by about 500 microns. The probe portions preferably have generally blunt or rounded ends to facilitate insertion thereof underneath a suture loop while preventing unintentional piercing or pricking of tender tissue.

The tip portion may further include at least one remover portion formed therewith which is preferably located adjacent the tip perimeter. The remover portion may be comprised of a series of generally aligned V-shaped notches which are sized and configured to grasp the suture. The remover portion may optionally be formed on any location on the suture cutter such as adjacent the opening. However, it is contemplated that the remover portion may also be formed on a portion of the cutting blade. Removal of severed sutures is facilitated through the engagement of the suture knot with the V-shaped notches of the remover portion. In this manner, severed sutures may be disengaged from the tissue of the patient by upwardly urging or lifting the knotted suture until the suture is completely dislodged or removed from the tissue.

The suture cutter may be formed from any suitable material such any plastic and/or metallic material or any combination thereof. Such material may further be a biocompatible material. It is contemplated that the suture cutter may be formed as an integral unitary structure comprising both the handle portion and the tip portion. In this manner, the suture may be economically mass-produced and therefore may warrant disposal following a single use in order to eliminate the risk of infection such as may be caused by transfer of bacteria or tissue between patients. Additionally, the suture cutter may be formed with the tip portion being disposably mountable on a reusable handle portion.

### BRIEF DESCRIPTION OF THE DRAWINGS

These as well as other features of the present invention will become more apparent upon reference to the drawings wherein:

FIG. 1 is a perspective view of a suture cutter of the present invention illustrating a user holding the suture cutter at a preferred 45° angle relative to suture-bearing tissue in preparation of severing of the sutures;

FIG. 2A is a plan view of a tip portion of the suture cutter and illustrating an arcuate cutting blade formed within an interior of the tip portion and further illustrating a remover portion formed along a side of the tip portion;

FIG. 2B is a plan view of the tip portion in an alternative embodiment showing the remover portion integrally formed along a portion of the cutting blade;

FIG. 3 is an end view of the tip portion illustrating a pair of opposing probe portions defining an opening therebetween and through which sutures may enter and engage the cutting blade;

FIG. 3a is a plan view of the tip portion in an alternative embodiment wherein the cutting blade is comprised of a center blade and a pair of side blades disposed on opposite sides of the center blade;

FIG. 3b is an end view of the tip portion similar to that which is shown in FIG. 3a but further including a protective tip extending across a forward edge of the center blade;

FIG. 3c is a plan view of the tip portion wherein the cutting blade is comprised of a center blade extending to one side of the opening with a side blade being disposed on an opposite side of the opening;

FIG. 4 is a plan view of the tip portion in a pull-back version of the suture cutter illustrating a forward blade disposed on the rearwardly extending probe portion;

FIG. 4a is a plan view of the tip portion in a further embodiment of the pull-back version further including an aft blade disposed opposite the forward blade;

FIG. 5 is a plan view of the tip portion in a further embodiment of the pull-back version shown in a symmetrical embodiment;

FIG. 6 is a plan view of the tip portion in a further embodiment of the suture cutter having an outer blade disposed in cantilevered relationship to an inner blade;

FIG. 6a is a plan view of the tip portion similar to that shown in FIG. 6 and illustrating a suture engaged between cutting edges of the inner and outer blades;

FIG. 6b is a plan view of the tip portion illustrating the suture cutter in a symmetrical version of the configuration shown in FIG. 6;

FIG. 6c is a section cut taken along lines 6c-6c of FIG. 6b and illustrating the inner and outer blades disposed in space relationship to define a blade gap therebetween;

FIG. 6d is a section cut taken along lines 6d-6d of FIG. 6b and illustrating cutting edges of the inner and outer blades being in direct engagement with one another;

FIG. 6e is a section cut taken along lines 6e-6e of FIG. 6b and illustrating a flange portion disposed on a tip perimeter of the suture cutter;

FIG. 6f is a plan view of the suture cutter similar to that shown in FIG. 6b and further including a protective cover disposed over the tip portion;

FIG. 7a is an exploded perspective view of the suture cutter illustrating a known surgical blade to which the tip portion may be removably attached;

FIG. 7b is a perspective view illustrating the suture cutter connected with the handle portion;

FIG. 8a is a plan view of the suture cutter in a configuration similar to that illustrated in FIGS. 3a and 3b and further including a neck portion configured for engagement to a handle portion of the known surgical blade handle;

FIG. 8b is a plan view of the tip portion configured similar to that illustrated in FIG. 3 and having the neck portion extending rearwardly therefrom;

FIG. 8c is a plan view of the tip portion in a configuration similar to that illustrated in FIG. 4 and having the neck portion connected thereto;

FIG. 8d is a plan view of the tip portion in a configuration similar to that illustrated in FIG. 4a;

FIG. 8e is a plan view of the tip portion in a configuration similar to that illustrated in FIG. 5;

FIG. 8f is a plan view of the tip portion in a configuration illustrated in FIG. 6; and

FIG. 8g is a plan view of the tip portion in a configuration similar to that illustrated in FIG. 6a.

### DETAILED DESCRIPTION

Referring now to the drawings wherein the showings are for purposes of illustrating the present invention and not for purposes of limiting the same, shown in the figures is a suture cutter 10 which is specifically adapted to cut and remove stitches and/or sutures 12 such as from the surface tissue 16 or skin of a patient in a disposable combination medical instrument. Advantageously, the suture cutter 10 includes a cutting blade 28 which is formed within an interior of the tip portion 22 and which is specifically configured to reduce or eliminate the risk of injury to a user or a patient during suture 12 cutting and removal.

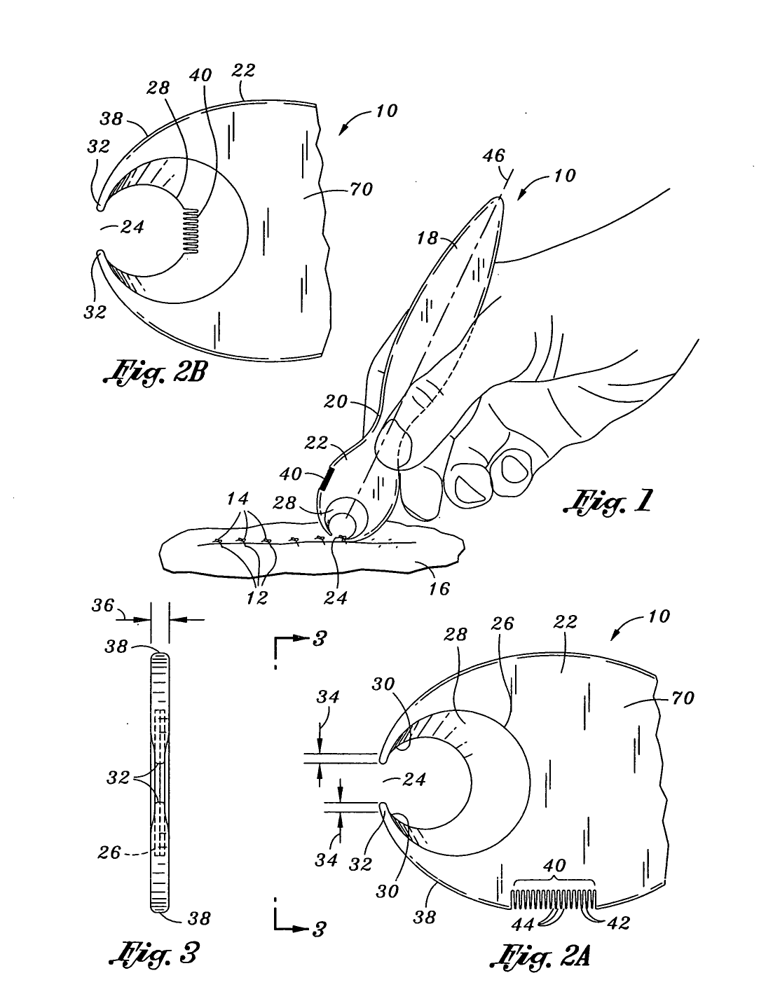

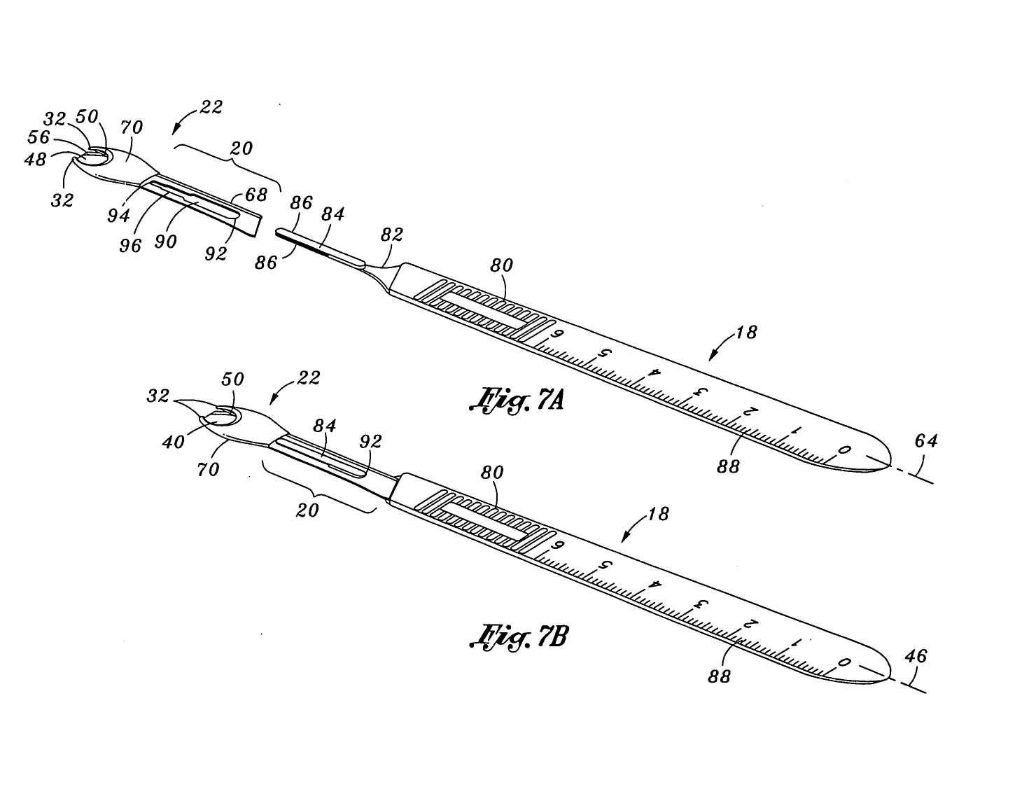

In its broadest sense, the suture cutter 10 comprises the tip portion 22 which may be integrally formed with or separately attachable to a handle portion 18. In FIG. 1, the suture cutter 10 is shown in perspective view as a unitary structure wherein a user may conveniently hold the suture cutter 10 in an optimal orientation of 45 degrees relative to the patient's skin 16. As can be seen, the handle portion 18 may be configured as an elongate and generally straight member which defines a tool axis 46. The handle portion 18 is preferably sized for convenient grasping and manipulation by a human hand. In this regard, it is contemplated that the handle portion 18 may have a generally rounded cross-sectional shape as shown in FIG. 1 or, alternatively, may be configured as a generally flat or planar shape as shown in FIGS. 7a and 7b. In this regard, the handle portion 18 is preferably provided in any suitable size and shape which provides an ergonomically shaped configuration.

When provided in the generally rounded cross-sectional shape, the handle portion 18 is preferably configured to allow for manipulation and/or rotation of the suture cutter 10 about the tool axis 46 into any angular orientation. In this manner, a user may better effectuate insertion of the cutting mechanism for subsequent removal of the suture 12. It is contemplated that the handle portion 18 may include surface features such as a gripping portion 80, textured bumps or a uniform gripping pattern extending over a substantial length of the handle portion 18. Such surface features may enhance manipulation of the suture cutter 10 during use.

Referring to FIG. 1, the suture cutter 10 is shown with the handle portion 18 integrated with the tip portion 22 and which further includes a neck portion 20 connecting the handle portion 18 to the tip portion 22. The tip portion 22 is the operative end of the suture cutter 10. The neck portion 20 may have a generally rounded or reduced cross-sectional area as compared to the cross-sectional area of the handle portion 18 and/or tip portion 22. Such reduced cross-sectional area facilitates gripping of the structure for manipulation between a forefinger and a thumb of the user while the remainder of the handle portion 18 may rest upon or within the user's palm or remainder of the user's fingers. It should be noted that the neck portion 20 is an optional feature of the suture cutter 10 and may be altogether omitted such that the handle portion 18 is directly connected to the tip portion 22.

Ideally, the handle portion 18 and/or neck portion 20 are formed and shaped in such a manner to be usable by either a left-handed or right-handed individual. In this regard, the handle portion 18 may be devoid of any specific left-hand or right-hand features such as finger indents or asymmetric shapes along its longitudinal axis. By providing a symmetrical handle shape, a variety of users may conveniently manipulate the handle portion 18 between the thumb and forefingers during operative use of the tip portion 22. Additionally, the omission of any asymmetric surface features on the handle portion 18 facilitates grasping thereof along any section of the handle portion 18. As can be seen in the figures, a free end of the handle portion 18 preferably has a tapered shape and a generally blunt or rounded tip.

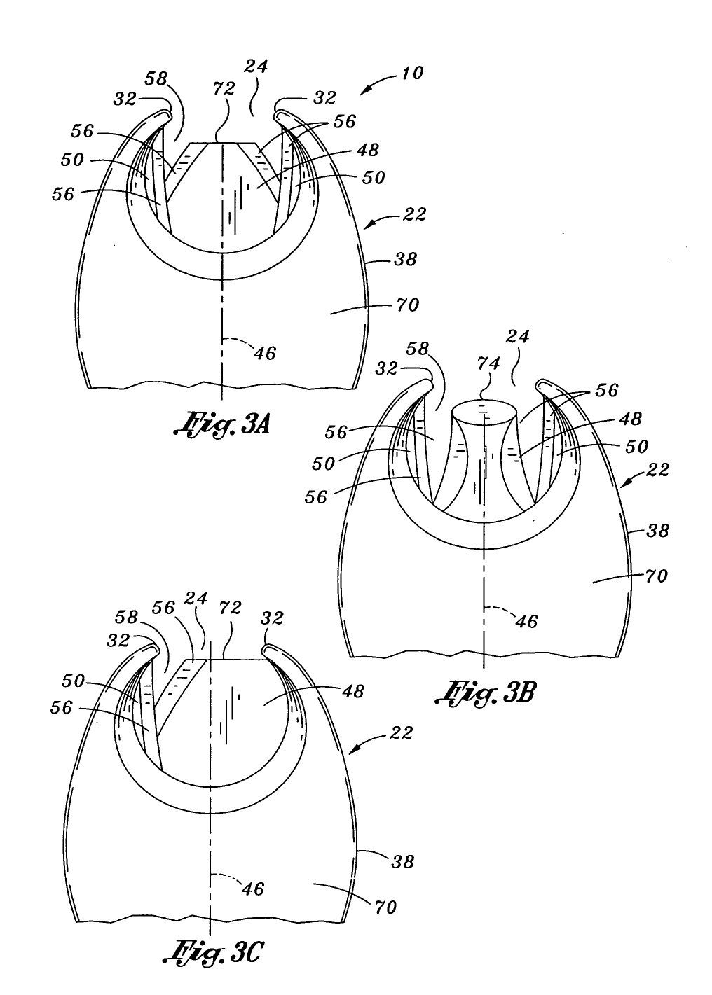

Referring now to FIGS. 2a and 2b, shown is the tip portion 22 which has a generally flattened or thin cross-sectional profile or shape. This thinned or flattened shape facilitates insertion of the tip portion 22 between the suture 12 loops to be cut and removed. The tip portion 22 preferably has a generally relatively small size that is complementary to the size of the suture 12 to be cut. Such small size of the tip portion 22 is desirable in preventing undue patient discomfort or pain during suture 12 removal. More specifically, by reducing the size of the tip portion 22, the extent to which relatively sensitive skin 16 must be depressed in order to insert the tip portion 22 between the suture 12 and the tissue 16 is reduced.

In FIGS. 2a and 2b, the tip portion 22 in one embodiment is shown in plan view wherein the perimeter 38 shape is illustrated having a preferred rounded shape and further illustrating a cutting blade 28 disposed within the interior of the tip portion 22. As was earlier mentioned, by configuring the tip portion 22 with the cutting blade 28 disposed therewithin, inadvertent contact is otherwise prevented as compared to a configuration wherein the cutting blade 28 is exposed. As shown in the configurations of FIGS. 2a and 2b, the cutting blade 28 is disposed adjacent the opening 24 which extends through the tip perimeter 38 and widens in the interior of the tip portion 22.

For the configuration shown in FIGS. 2a and 2b, at least a portion of the cutting blade 28 is arranged to face toward the opening 24 formed in the tip perimeter 38. As can be seen, the cutting blade 28 is preferably of an annular or concavely shaped member although a variety of alternative sizes and configurations are contemplated. For example, the cutting blade 28 may be configured in a general horseshoe shape or, alternatively, may have an orthogonally-configured section of cutting blade 28 as opposed to the arcuate shaped cutting blade 28 shown in the figures. As was earlier mentioned, the cutting blade 28 is oriented to generally face toward at least a portion of the opening 24 to thereby facilitate cutting of the sutures 12 passing into the opening 24.

The cutting blade 28 may be integrally formed with the tip portion 22 or it may be formed as a separate member. For the integral configuration shown in FIGS. 2a and 2b, the suture cutter 10 may be fabricated as a single component from metallic material in order to provide a durable and sharpened edge for effective cutting of the suture 12 by the cutting blade 28. Alternatively, the cutting blade 28 may be formed as a separate metallic or other suitably hard component and may be shaped in the form of a metallic insert that is then received within a slot or groove 86 formed in a protective cover 70. If provided as an insert, the cutting blade 28 may be retained by the receiving slot 26 formed within the protective cover 70 of the tip portion 22 and may be secured thereto by frictional engagement within the receiving slot 26. Alternatively, a suitable adhesive and/or mechanical fastening means or any combination thereof may be utilized to secure the cutting blade 28 to the protective cover 70.

Referring still to FIGS. 2a and 2b, the tip portion 22 may further include a pair of probe portions 32 which are sized to extend outwardly from the tip perimeter 38 on opposing sides of the opening 24. Each of the probe portions 32 preferably has a rounded or bulbous tip to minimize or prevent injury to the patient and/or the user. As can be seen in FIG. 2a, the tip portion 22 preferably extends slightly past opposing terminal ends 30 of the cutting blade 28 by a setback 34 amount. The setback 34 amount is preferably such that a suture 12 may readily pass through the opening 24 prior to direct engagement with the cutting blade 28. Although the cutting blade 28 and tip perimeter 38 generally converge in order to form the probe portion 32 on opposing sides of the opening 24, the setback 34 of the probe portion 32 relative to the terminal ends 30 provides a safety feature for the cutting blade 28 against accidental injury to a user or patient.

Preferably, each of the probe portions 32 is configured to extend slightly past an adjacent one of the terminal ends 30 of the cutting blade 28 and is preferably no greater than about 1000 microns for the setback 34 amount. Most preferably, the probe portions 32 protrude about 500 microns beyond an adjacent one of the terminal ends 30. As was earlier mentioned, the probe portions 32 are provided with generally rounded or bulbous tips in order to facilitate painless insertion of the cutting blade 28 underneath the suture 12 loop in order to avoid unintentionally piercing or pricking tender tissue 16 surrounding the suture 12.

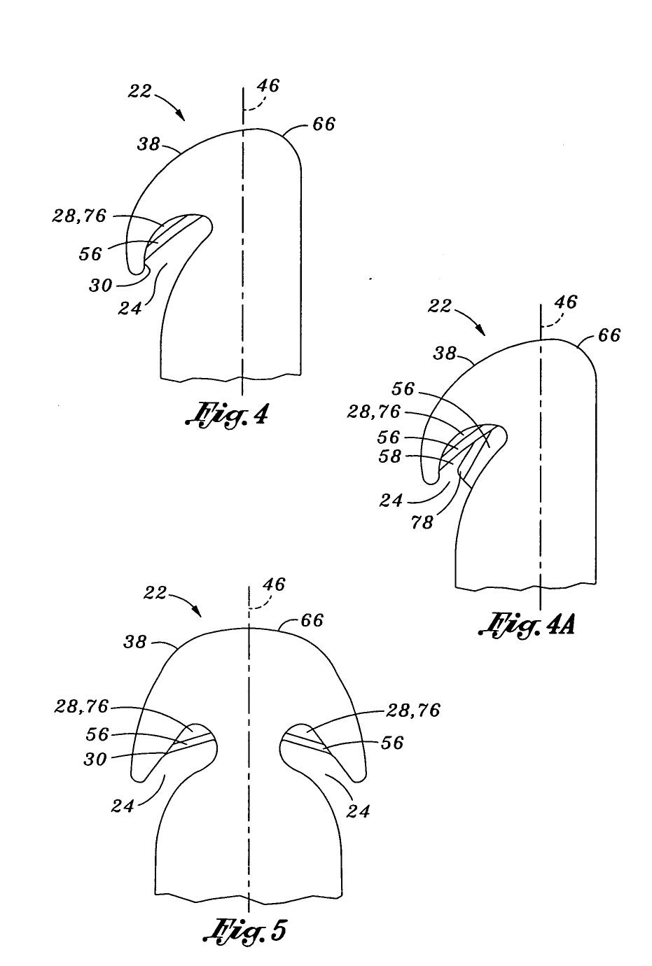

Referring briefly to FIG. 3, shown is an end view of the tip portion 22 illustrating its generally flat and thin cross-sectional shape. This low profile of the tip portion 22 facilitates insertion of the probe portions 32. Specifically, the tip portion 22 may be provided with a thickness 36 of preferably less than about 1.5 millimeters (mm) and, most preferably, has a thickness 36 of about 0.75 mm. As shown in FIGS. 2a and 3, the opposing probe portions 32 collectively define the width of an opening 24 of the tip portion 22. As can be seen in FIG. 3, the cross-sectional area or thickness 36 of the tip portion 22 at the probe portion 32 is preferably less than the cross-sectional area of the remainder of the tip portion 22. Such an arrangement facilitates easy insertion of the probe portion 32 between the suture 12 and the tissue 16 surrounding the suture 12.

Referring back to FIGS. 2a and 2b, the tip portion 22 may optionally include at least one remover portion 40 formed therewith and preferably located along the tip perimeter 38. As can be seen, the remover portion 40 may be comprised of a series of generally aligned V-shaped notches 42 which are specifically sized and configured to grasp the suture 12 for removal thereof following cutting; Although shown in FIG. 2a as being formed on the tip perimeter 38, the remover portion 40 may optionally formed on any other area of the suture cutter 10. For example, as shown in FIG. 2a, the remover portion 40 may be located along a tip perimeter 38 at a position that is generally 90 degrees relative to the opening 24. Furthermore, the remover portion 40 may be arranged so as to be generally parallel to the tool axis 46 which extends along a length of the suture cutter 10.

The placement of the remover portion 40 as shown in FIG. 2a is believed to be advantageous in that, following the severing of a series of sutures 12, the sutures 12 may then be easily removed by repositioning the tool axis 46 in general parallel orientation relative to the surface of the skin 16. The remover portion 40 has a generally comb-like configuration wherein the V-shaped notches 42 are generally aligned with one another and are formed by the tines 44. The user may then engage the suture 12 with a set of tines 44 formed by the V-shaped notches 42 which comprise the remover portion 40. In this manner, the V-shaped notches 42 may grasp the suture 12 for lifting out of the wound by the user. More specifically, because sutures 12 typically include a knot 14 that is created during installation of the sutures 12, removal of the suture 12 is facilitated by disengaging, lifting and pulling the knot 14 captured between the V-shaped notches 42.

Referring to FIG. 2b, shown is the suture cutter 10 in an alternative embodiment showing the tip portion 22 wherein the remover portion 40 is formed on at least a part of the cutting blade 28. As can be seen, the remover portion 40 may be positioned to face generally forward or at least be accessible by the opening 24 of the tip portion 22. Such positioning enhances severing of the sutures 12 wherein the suture cutter 10 may be first oriented at an approximate angle of about 45 degrees relative to the tool axis 46 with the patient's skin 16.

Following severing of one or a series of sutures 12, the suture cutter 10 may then be reoriented such that the tool axis 46 is moved into substantial parallel alignment to the patient's skin 16. The user may then pass the tool over the wound in order to engage the sutures 12 with the V-shaped notches 42. Once engaged, the sutures 12 may then be lifted out of the wound. Although the remover portion 40 is shown as being disposed upon the tip perimeter 38 in FIG. 2a or on the cutting blade 28 in FIG. 2b, the remover portion 40 may be located in any suitable area on the suture cutter 10 in order to facilitate convenient removal of the sutures 12.

Referring now to FIGS. 3a to 3c, shown is the suture cutter 10 wherein the tip portion 22 comprises a forward-facing opening 24 formed therein and which has at least a center blade 48 disposed within the interior of the tip portion 22. As can be seen in the figures, at least a portion of the center blade 48 is located within a general centralized area of the opening 24 and may extend to at least one of the sides of the opening 24. For example, as shown in FIG. 3c, the center blade 48 is shown as having a generally straight forward edge 52 facing the opening 24 with a majority of the opening 24 being occupied by a remainder of the center blade 48.

The cutting edge 56 is formed on a side of the center blade 48 and is formed at an angle in a slight arcuate shape extending toward a side blade 50. As can be seen in FIG. 3c, the side blade 50 is also disposed within the interior of the tip portion 22 adjacent a side of the center blade 48 and also has a cutting edge 56 associated therewith that converged toward the cutting edge 56 of the center blade 48. The configurations shown in FIGS. 3a-3c further illustrate the side blade 50 having a terminal end 30 disposed adjacent the probe portion 32. As can be seen, the probe portion 32 is disposed on a side of the opening 24 adjacent the terminal end 30 of the side blade 50 and the probe portion 32 extends slightly past the terminal end 30 in the same manner and by the same preferable amounts as described above for the versions of the suture cutter 10 illustrated in FIGS. 1-2a. As can be seen, the cutting edges 56 of the center and side blades 48, 50 collectively define a blade gap 58 which is generally open in an area adjacent the opening 24 and which narrows in width along an aftward direction.

Preferably, the blade gap 58 narrows such that the cutting edges 56 eventually converge in order to allow effective severing of sutures 12 which pass through the opening 24 and enter the blade gap 58. In the embodiments shown in FIGS. 3a and 3b, a pair of the side blades 50 are disposed on opposing sides of the center blade 48. Each one of the side blades 50 has a terminal end 30 with a pair of probe portions 32 being disposed on opposing sides of the opening 24 adjacent respective ones of the terminal ends 30. In the manner as was earlier described, each of the probe portions 32 has a generally rounded or bulbous tip and extends slightly past an adjacent one of the terminal ends 30 such that the probe portion 32 collectively define the width of the opening 24.

The suture cutter 10 shown in FIGS. 3a-3c has the opening 24 being generally centered along the tool axis 46 of the suture cutter 10 such that a user may simply move the suture cutter 10 in a forward direction in order to capture and cut sutures 12 passing through the opening 24 and engaging the cutting edges 56 of the side and center blades 50, 48. However, it is contemplated that the opening 24 and, hence, the side and center blades 50, 48 may be offset from the tool axis 46 to one side or the other in order to more effectively cut the sutures 12 depending on the characteristics of the user as well as the geometry of the area of tissue 16 from which the sutures 12 are to be removed.

As shown in FIGS. 3a-3c, the tip portion 22 may be configured with a protective cover 70 having the cutting blades 28 (i.e., the center and side blades 48, 50) protruding thereout. More specifically, in the manner described above for the suture cutter 10 of FIGS. 1-2a, the suture cutter 10 of FIGS. 3a-3c may be configured such that the center and side blades 48, 50 are fabricated as metallic inserts which are frictionally and/or otherwise retained within a receiving slot 26 formed in the protective cover 70. For example, as shown in FIG. 3, the protective cover 70 may include the receiving slot 26 centered within a tip width of the tip portion 22. The protective cover 70 may be formed of a polymeric material as a safety feature to prevent exposure of otherwise hard and metallic edges of the cutting blades 28 regardless of whether cutting edges 56 or other parts of the cutting blades 28 are exposed.

For such purposes, the protective cover 70 preferably has generally rounded outer edges such as that shown in FIG. 3 in order to minimize the risk of inadvertent piercing or slicing of the patient's and/or user's skin 16. A further safety feature may be provided in the form of a protective tip 74 disposed over the forward edge 52 of the center blade 48 as shown in FIG. 3b. Such protective tip 74 may be preferably formed of a biocompatible polymeric material. Such protective tip 74 preferably has a generally rounded forward area or side such that the forward end of the cutting edge 56 of the center blade 48 is covered.

The configuration of the opening 24 defined by a gap between the probe portions 32 for the configuration shown in FIGS. 3a-3c is similar to that illustrated in FIGS. 2a and 2b and as was described above. More specifically, the probe portions 32 preferably extend slightly past the terminal ends 30 by an amount no greater than about 1000 microns for the setback 34 amount. Most preferably, the probe portion 32 protrudes about 500 microns beyond the terminal end 30. As was earlier mentioned, the probe portions 32 are provided with generally rounded or bulbous tips to facilitate insertion of the probe portion 32 underneath a suture 12 loop prior to sliding the suture cutter 10 forward in order to engage the suture 12 with the cutting edges 56 of the side and/or center blades 50, 48.

Following severing of the suture 12, the remover portion 40 may be provided on the configuration of the suture cutter 10 shown in FIGS. 3a and 3c similar to that which was shown in FIGS. 2a and 2b and which was described above. Furthermore, the geometry of the tip portion 22 of the configurations shown in FIGS. 3a-3c is similar to that described above for the configurations of FIGS. 2a-2b wherein the probe portions 32 have a thickness or cross-sectional area that is generally less than the thickness 36 of the remainder of the tip portion 22. For example, the tip portion thickness 36 is preferably less than about 1.5 mm and, most preferably, has a thickness 36 of about 0.75 mm.

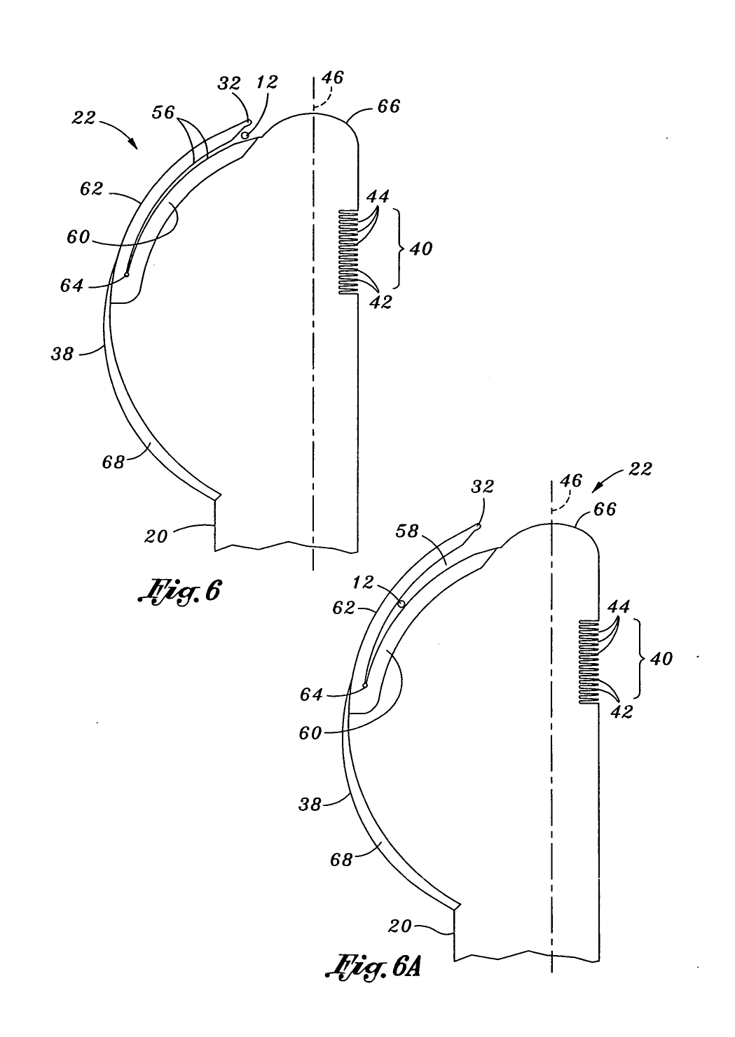

Referring now to FIGS. 4-5, shown is the suture cutter 10 in an alternative embodiment wherein the tip portion 22 is provided with an aft-facing opening 24 formed on a lateral side of the tip portion 22. The tip portion 22 is provided with the cutting blade 28 which is disposed within the interior of the tip portion 22 accessible to the opening 24. As can be seen in reference to FIGS. 4 and 5, the cutting blade 28 is mounted on a forward end of the opening 24 such that the cutting blade 28 is referred to as a forward blade 76. In this regard, the opening 24 may be provided with a pair of blades each having a cutting edge 56 to collectively define a blade gap 58 by which a suture 12 may be wedged in between during a pulling-back motion of the suture cutter 10 to effectuate severing of the suture 12. For the forward blade 76, the probe portion 32 is disposed adjacent the terminal end 30 thereof such that the probe portion 32 extends slightly past the outermost end of the cutting edge 56 in the same manner as described above for the configurations shown in FIGS. 1-3c.

For the configuration of the suture cutter 10 shown in FIGS. 4 and 5, it can be seen that the cutting edge 56 of the forward blade 76 is obliquely oriented with reference to the tool axis 46. In this manner, effective severing of the suture 12 may be facilitated by pulling back such that the probe portion 32 may be slid between the tissue 16 and the suture 12 extending through the tissue 16. By sizing the probe portion 32 to have a generally reduced cross-sectional area or thickness, the probe portion 32 may be more easily inserted thereunder without causing undue pain or discomfort to the patient in the sensitive area of the patient wound. Further retracting the suture cutter 10 then causes the suture 12 to ride up along the cutting edge 56 of the forward blade 76 until the pressure exerted by the cutting edge 56 severs the suture 12. A series of sutures 12 may be cut in this manner by simply slightly depressing the probe portion 32 to engage the suture 12 in a sequential manner followed by sequential cutting of the sutures 12.

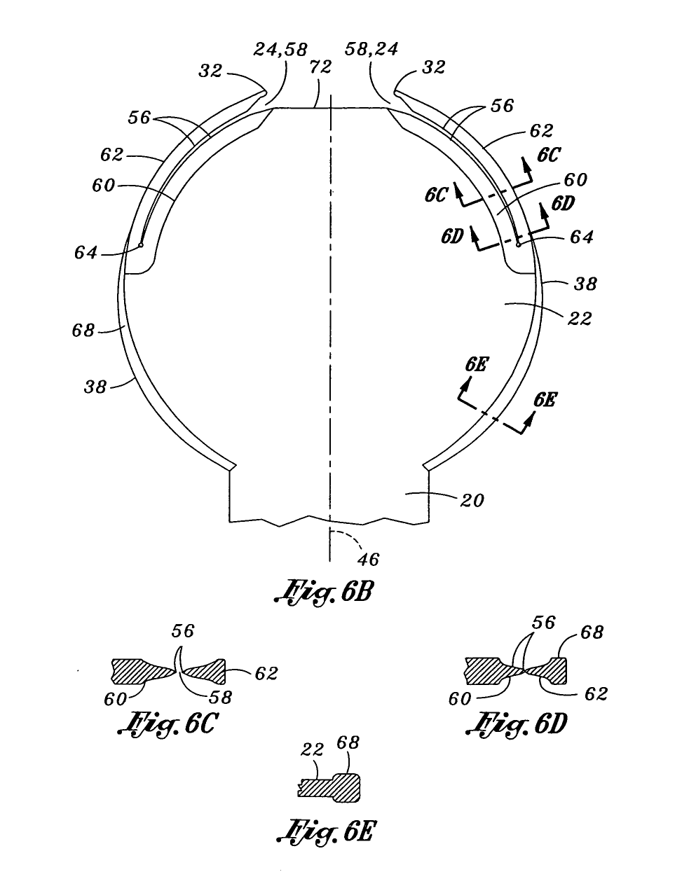

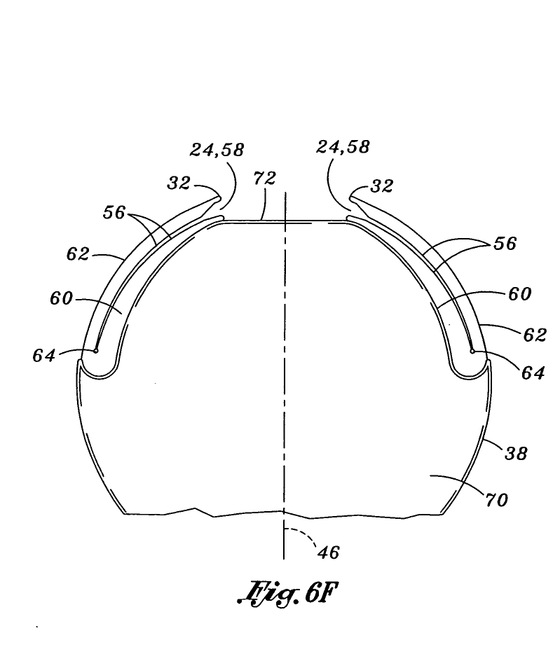

Referring now to FIG. 4a, providing the aft blade 78 in combination with the forward blade 76 may reduce the extent of upward pulling of the suture 12 away from the skin 16 because the suture 12 may be collectively severed by the cutting edges 56 of the forward and aft blades 78 without the necessity for the suture 12 to laterally travel inward into the interior of the tip portion 22 as may be the case with the configurations shown in FIGS. 4 and 5. Referring now to FIGS. 6 and 6a, shown is the suture cutter 10 in an additional embodiment wherein the tip portion 22 includes an inner blade 60 formed along an integral width the tip perimeter 38. The inner blade 60 has a terminal end 30 disposed at its forward end. The tip portion 22 further includes an outer blade 62 which extends from the tip perimeter 38 and is preferably disposed adjacent the inner blade 60 such that the inner and outer blades 60, 62 have cutting edges 56 which collectively define a blade gap 58.

As was described above for FIGS. 3a-3c and 4a, each of which has a pair of blades defining a blade gap 58, the embodiment shown in FIGS. 6, 6a, 6b and 6f facilitate severing of the suture 12 utilizing the same principal of operation wherein the suture 12 is severed due to a combination of cutting forces exerted by the cutting edges 56 of the pair of adjacently disposed blades. The cutting pressure increases due to a decrease in the blade gap 58 as the suture 12 travels further thereinto. As can be seen in FIGS. 6 and 6a, the inner and outer blades 60, 62 have an arcuate shape which extends from the generally arcuate tip perimeter 38. As such, the inner and outer blades 60, 62 are fabricated such as by machining from a single metallic material such as surgical steel. The tip portion 22 may be integrally formed or detachably removable from the handle portion 18 in a manner as will be described in greater detail below.

Referring still to FIGS. 6-6f, it can be seen that the outer blade 62 has a generally thin cross-section. In this manner, the amount by which the suture 12 is pulled away from the tissue 16 is minimized. Rather, the configurations of FIGS. 6-6f facilitate horizontal or lengthwise (i.e., along the tool axis 46) pulling of the suture 12 during severing operations. By minimizing such upward or pulling movement of the suture 12, pain and discomfort is minimized. Upward movement and subsequent severing of the suture 12 is facilitated by flexing of the outer blade 62 relative to the inner blade 60.

Toward this end, the tip perimeter 38 of the tip portion 22 may include an I-beam type of construction to increase the relative stiffness and strength of the outer blade 62 in resisting its outward flexion relative to the remainder of the tip portion 22. For example, as shown in FIG. 6e, the tip perimeter 38 of the tip portion 22 is provided with a flange portion 68 shown as having a generally increased thickness relative to a remainder of the tip portion 22. FIG. 6d illustrates the flange portion 68 extending into the outer blade 62 at which point a cutting edge 56 is formed thereon. Likewise, FIG. 6d illustrates that the same location wherein the cutting edge 56 of the inner blade 60 is initiated. Moving further along, the cross-section of FIG. 6c illustrates a reduction or elimination of the increased thickness of the flange portion 68 in the outer blade 62 in order to facilitate outward flexion of the outer blade 62 to minimize pulling of the suture 12 away from the patient's skin 16.

Such outward flexion may induce stresses between the inner and outer blades 60, 62. For this reason, a small relief 64 is provided at the apex of the cutting edges 56 of the inner and outer blades 60, 62 and is configured as a generally rounded hole in order to provide a stress riser between the inner and outer blades 60, 62. Likewise, the opposite set of inner and outer blades 60, 62 on the tip portion 22 are also provided with a relief 64 to minimize stress cracking that may occur as a result of excess bending moment exerted by the upward pulling of the suture 12 from the outer blade 62.

The thickness of the flange portion 68 and its profile in plan view as shown in FIG. 6b is preferably such as to provide an appropriate amount of tension and strength to the inner and outer blades 60, 62. For this reason, the flange portion 68 is shown as being tapered and eventually terminating at less than a third a length of the outer blade 62. However, it should be noted that the profile (i.e., thickness and cross-sectional area) may be provided in various sizes and tapers. Due to the unique configuration of the suture cutter 10 shown in FIGS. 6a-6f, relatively small diameter sutures 12 may be readily cut by the suture cutter 10. It should also be noted that the suture cutter 10 may be provided in non-symmetric configurations such as that shown in FIGS. 6 and 6a where the general geometric and operational characteristics are similar as to those of the configurations shown in FIGS. 6b and 6f. Furthermore, the remover portion 40 may optionally be provided along a generally straight side of the tip portion 22 with the forward end thereof having a generally rounded nose 66.

For each of the configurations of the tip portion 22 shown from FIGS. 2a-2b, 3a-3c, 4-5 and 6-6f, the tip portion 22 may be configured to be detachably removable from a separately formed oblong or elongate planar handle portion 18 in a surgical blade handle configuration well known in the art. More specifically, U.S. Pat. No. 4,617,738 discloses and illustrates a conventional handle having a tang 82 extending outwardly therefrom. The tang 82 includes a raised portion 84 having a pair of grooves 86 of controlled dimension on a forward end thereof. The grooves 86 are specifically adapted to engage an engagement slot 90 formed within the neck portion 20 which may be integrally formed with the tip portion 22 in any number of configurations described above. The engagement slot 90 includes a generally enlarged or wide area at the proximal end 92 with a generally tapering or narrowed portion at a distal end 94 thereof. Importantly, a transition portion 96 is provided intermediate the distal end 94 and proximal end 92 which is specifically adapted to snugly engage the pair of grooves 86 disposed on opposing sides of the raised portion 84 of the tang 82.

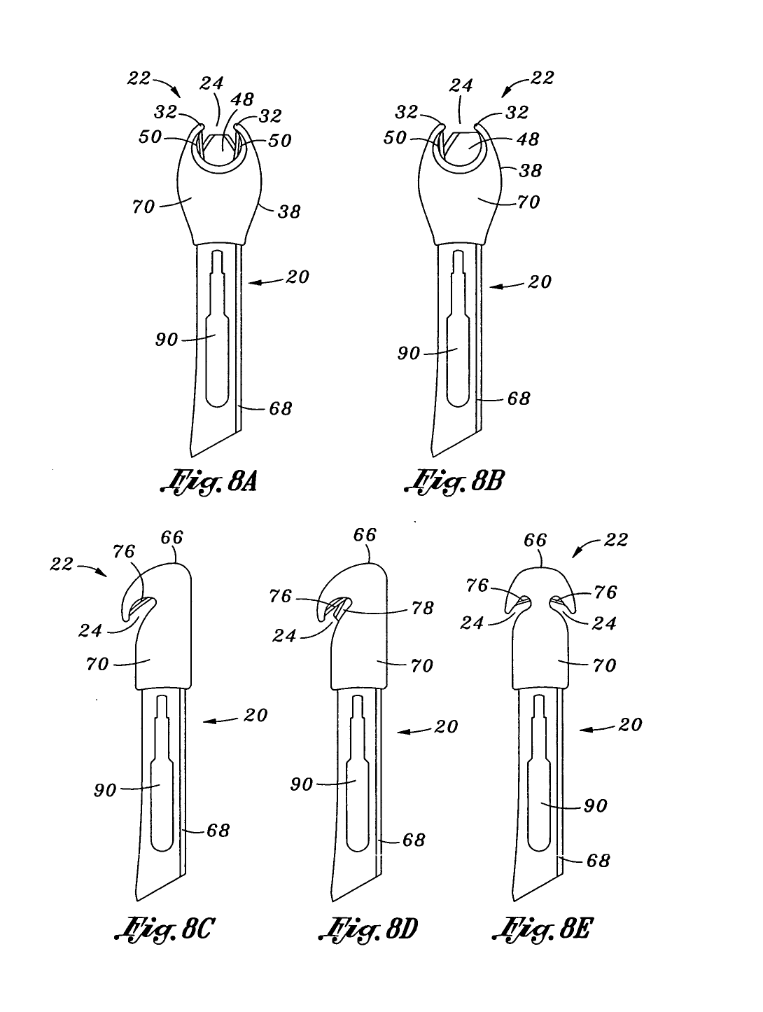

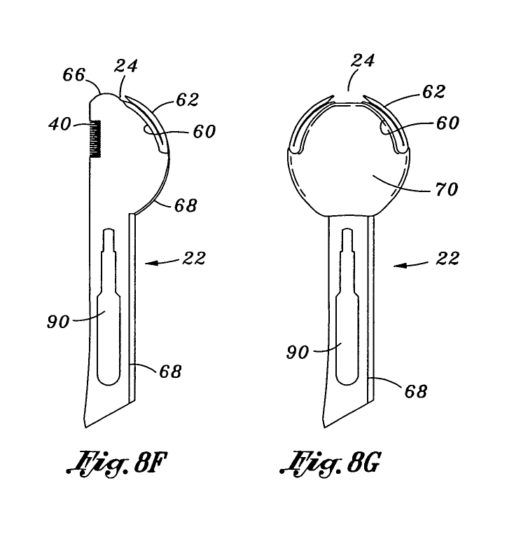

As described in detail in U.S. Pat. No. 4,617,738, the entire of contents of which are expressly incorporated by reference herein, the various configurations of the tip portion 22 of the suture cutter 10 as shown in FIGS. 8a-8g may be conveniently and quickly mounted and removed from the handle portion 18. Such engagement of the suture cutter 10 is facilitated by passing a forward part of the raised portion 84 of the handle portion 18 through the engagement slot 90 of the neck portion 20. The pair of grooves 86 on opposing sides of the raised portion 84 are then received by the transition portion 96 until the proximal end 92 of the neck portion 20 may be extended over an aft end of the raised portion 84. In this manner, the tip portion 22 may be removably secured in a disposable manner to the handle portion 18 as shown in FIG. 7b.

By fabricating the tip portion 22 as a disposable part of the suture cutter 10, the risk of infections otherwise caused by transfer of bacteria or germs to the sutured area may be minimized. In addition, fabricating the tip portion 22 as a separate component reduces the risk of infection that may be otherwise caused by the transfer of tissue 16 or fluid from one patient to another. A further advantage of configuring the tip portion 22 separate from the handle portion 18 is associated with the elimination of the need for sterilization of the tip portion 22 following each use. Therefore, provided is a disposable medical instrument comprised of a unitary combination medical instrument specifically adapted for cutting and/or severing of stitches or sutures 12 while allowing for subsequent removal and extraction of the sutures 12.

As was earlier mentioned, the tip portion 22 may be fabricated of a metallic material such as surgical steel or any other suitable material upon which a cutting edge 56 may be formed in the cutting blade 28. In addition, the tip portion 22 may include a protective cover 70 fabricated of a polymeric or other suitable material or any combination thereof in order to encase otherwise sharp edges of the cutting blade 28 to prevent inadvertent pricking or cutting of the patient or tissue 16. Ideally, a suitable material such as a biocompatible material may be used. In the interest of economy, the suture cutter 10 may be fabricated as an integration of the tip portion 22 with the handle portion 18 and, optionally, the neck portion 20. If fabricated of a polymeric material, a receiving slot 26 may be provided in the tip portion 22 for receiving a metallic insert comprising the cutting blade 28. When constructed as a unitary structure, the suture cutter 10 may be effectively mass produced at low cost.

The operation of the suture cutter 10 of above-described embodiments will now be described with reference to the drawings wherein FIG. 1 illustrates a user grasping the handle and neck portions 18, 20 between the forefinger and thumb during suture 12 cutting operations. As was earlier mentioned, an optimal orientation at which the suture cutter 10 may be held is at an angle of about 45° which is believed to facilitate the severing of single or multiple sutures 12. As can be seen in FIG. 1, severing is initiated by inserting one of the probe portions 32 between the suture 12 and the patient's skin 16 by slightly depressing the patient's skin 16 to the minimum unnecessary depression of tender tissue 16.

Following insertion of the probe portion 32 under the suture 12, the suture 12 passes through the opening 24 and is brought into close proximity to the cutting blade 28. Further movement of the handle portion 18 brings the suture 12 into direct contact with the cutting blade 28 such that the cutting blade 28 ultimately severs the suture 12. Multiple sutures 12 may be sequentially severed by moving the handle portion 18 along the length of the wound such that the probe portion 32 engages each suture 12 followed by severing thereof and repeating the sequence until all sutures 12 are severed.

Removal of the severed sutures 12 may be effectuated by repositioning the handle portion 18 such that the remover portion 40 is oriented to engage the sutures 12 when the suture cutter 10 is moved toward the severed sutures 12. Ideally, the sutures 12 are severed such that the knot 14 of the suture 12 is generally extended above the patient's skin 16 to a sufficient amount such that the remover portion 40 may be engaged without contacting sensitive tissue 16. The V-shaped notches 42 of the remover portion 40 are moved into the suture 12 until the suture 12 and/or the suture knot 14 is engaged 42. Once sufficiently engaged to the V-shaped notches 42, the suture cutter 10 may be moved in a direction generally away from the patient's skin 16 in order to lift the complete suture 12 out of the tissue 16. The remaining sutures 12 may be removed in the same manner.

Additional modifications and improvements of the present invention may be also apparent to those of ordinary skill in the art. Thus the particular combination of parts described and illustrated herein is intended to represent only certain embodiments of the present invention and is not intended to serve as limitation of alternative devices within the spirit and scope of the invention.

What is claimed is:

1. A suture cutter for cutting a suture, comprising:
a tip portion having an opening formed therein and including:
a cutting blade disposed within an interior of the tip portion and having opposing terminal ends;
a pair of probe portions disposed on opposing sides of the opening adjacent respective ones of the terminal ends, each one of the probe portions extending slightly past an adjacent one of the terminal ends such that the probe portions define the opening.

2. The suture cutter of claim 1 further comprising:
a handle portion;
wherein the tip portion is configured to be removably attachable to the handle portion.

3. The suture cutter of claim 2 wherein the tip portion is integrally formed with the handle portion such that the suture cutter comprises a unitary structure.

4. The suture cutter of claim 3 wherein:
the handle portion and tip portion are formed of polymeric material;
the cutting blade being formed as an insert of metallic construction and which is retained within the tip portion.

5. The suture cutter of claim 1 wherein the tip portion includes a protective cover disposed thereover and being configured such that the cutting blade protrudes thereout.

6. The suture cutter of claim 5 wherein the protective cover is formed of polymeric material and the cutting blade is formed of metallic material.

7. The suture cutter of claim 1 further including at least one remover portion disposed on a tip perimeter of the tip portion and having a comb-like configuration.

8. The suture cutter of claim 7 wherein the remover portion is comprised of a plurality of V-shaped notches.

9. A suture cutter for cutting a suture, comprising:
a tip portion having a forward-facing opening formed therein and including:
a center blade disposed within an interior of the tip portion;
at least one side blade disposed within an interior of the tip portion adjacent a side of the center blade, the side blade having a terminal end;
at least one probe portion disposed on a side of the opening adjacent the terminal end, the probe portion extending slightly past the terminal end;
wherein the center and side blades collectively define a blade gap narrowing in width from the opening.

10. The suture cutter of claim 9 further comprising:
a handle portion;
wherein the tip portion is configured to be removably attachable to the handle portion.

11. The suture cutter of claim 10 wherein the tip portion is integrally formed with the handle portion such that the suture cutter comprises a unitary structure.

12. The suture cutter of claim 9 wherein:
the center blade has a pair of the side blades disposed on opposing sides of the center blade;
each one of the side blades having a terminal end;
a pair of probe portions being disposed on opposing sides of the opening adjacent respective ones of the terminal ends, each one of the probe portions extending slightly past an adjacent one of the terminal ends such that the probe portions define the opening.

13. The suture cutter of claim 9 wherein the center blade has a forward edge with a protective tip disposed thereover.

14. A suture cutter for cutting a suture, comprising:
a tip portion having an aft-facing opening formed on a side of the tip portion, the tip portion including:
a cutting blade disposed within an interior of the tip portion and having a terminal end; and
a probe portion disposed adjacent the terminal end and extending slightly therepast.

15. The suture cutter of claim 14 wherein a pair of the cutting blades are disposed on opposing sides of the tip portion.

16. The suture cutter of claim 14 wherein the cutting blade is comprised of a forward blade disposed adjacent an aft blade, the forward and aft blades having cutting edges collectively defining a blade gap narrowing in width from the opening.

17. The suture cutter of claim 14 further comprising:
a handle portion;
wherein the tip portion is configured to be removably attachable to the handle portion.

18. The suture cutter of claim 17 wherein the tip portion is integrally formed with the handle portion such that the suture cutter comprises a unitary structure.

19. A suture cutter for cutting a suture, comprising:
a tip portion defining a tip perimeter and including:
an inner blade formed on a side of the tip portion and having a terminal end;
an outer blade extending from the tip perimeter and being disposed adjacent the inner blade, the inner and outer blades collectively defining a blade gap for receiving a suture therewithin.

20. The suture cutter of claim 19 wherein a pair of the inner and outer blades are disposed on opposing sides of the tip portion.

21. The suture cutter of claim 19 further comprising:
a handle portion;
wherein the tip portion is configured to be removably attachable to the handle portion.

22. The suture cutter of claim 19 wherein the tip portion is integrally formed with the handle portion such that the suture cutter comprises a unitary structure.
